(19) United States
(12) Plant Patent Application Publication
(10) Pub. No.: US 2015/0342102 P1
(43) Pub. Date: Nov. 26, 2015

(54) Lemon tree named 'SUMMER PRIM'

(50) ;

(71) Applicant: Juan Antonio Gómez MARIN, Murcia (ES)

(72) Inventor: Juan Antonio Gómez MARIN, Murcia (ES)

(21) Appl. No.: 14/545,406

(22) Filed: May 1, 2015

(30) Foreign Application Priority Data
May 20, 2014 (ES) 20145150

(51) Int. Cl. A01H 5/00

(52) U.S. Cl. PLT/201

(57) ABSTRACT

A new and distinct cultivar Citrus limon (L.) Brum.f. is provided which displays a substantially different time for initial fruit harvest than the standard ‘Primofiori’ cultivar (non-patented in the United States). The attractive juicy fruit of the new cultivar commonly is initially ready for harvest in about the middle of May. This follows greatly the typical November initial harvest time for the ‘Primofiori’ cultivar. The new cultivar is a diploid, and displays a relatively dense spreading growth habit. The supply of the fresh fruit market for consumption, use in beverages, and in cooking is facilitated at an atypical season of the year by the new cultivar.

### BOTANICAL/COMMERCIAL CLASSIFICATION

Citrus limon (L.) Brum.f./Lemon Tree

### VARIETAL DENOMINATION

cv. Summer Prim

### SUMMARY OF THE INVENTION

The new and distinct late-ripening lemon cultivar of the present invention was discovered while growing in a cultivated area of the Murica region of Spain while present among standard ‘Primofiori’ lemon trees (non-patented in the United States). A single plant of the new cultivar was found, was carefully preserved, and thereafter was observed to confirm its distinctive combination of characteristics. Had the new cultivar of the present invention not been discovered and preserved it would have been lost to mankind. This new cultivar is believed to be a spontaneous mutation of the ‘Primofiori’ lemon tree of unknown causation.

It was found that the new Lemon cultivar of the present invention:

(a) is a diploid,

(b) forms attractive fruit which when compared to the ‘Primofiori’ cultivar displays a substantially later season of maturity, and,

(c) displays a relatively dense spreading growth habit. The new cultivar has been asexually reproduced at the Murcia region of Spain by grafting on standard ‘sour orange’ rootstock (non-patented in the United States). The combination of characteristics, including the consistent production of late maturing fruit, has been shown to be stable and is reliably transmitted to succeeding generations following such asexual reproduction. Accordingly, the new cultivar reproduces in a true-to-type manner by such asexual reproduction.

The fruit quality of the new cultivar has been found to be substantially similar to that of the standard ‘Primofiori’ cultivar.

The new cultivar can be readily distinguished from ‘Primofiori’ lemon in view of its extremely later time for the harvest of mature fruit. For instance, at the Murcia region of Spain the fruit of the ‘Primofiori’ lemon commonly is initially of a maturity suitable for harvest in November. However, the fruit of the new cultivar commonly is of a maturity suitable for harvest approximately six months later (i.e., during the following middle of May) when grown under comparable conditions in the Northern Hemisphere. Harvest commonly is carried out from about mid-May until about mid-July. The fruit of the new cultivar also initially matures at a substantially later time than the ‘Verna’ cultivar (non-patented in the United States).

Accordingly, growers of the new cultivar are able to supply the commercial market with quality freshly-picked lemon fruit at a different season of the year than growers of the ‘Primofiori’ lemon. The time of initial fruit maturity for new cultivar is displayed at a different time when fresh lemons continue to be in demand for purchase by consumers. Growers of the new cultivar thereby are enabled to benefit significantly from the atypical season of fruit maturity that is reliably manifest by the presently-claimed new lemon plant. It follows that those growing the standard ‘Primofiori’ lemon are unlikely to be able to offer quality freshly-picked mature fruit at the same time of the year.

The new plant of the present invention has been named ‘Summer Prim’.

### BRIEF DESCRIPTION OF THE PHOTOGRAPHS

The accompanying photographs show, as nearly true as is reasonably possible to make the same in color illustrations of this character, a typical tree of the new cultivar, as well as a comparison of the whole and sectioned fruit of the new cultivar with that of the ‘Primofiori’ and ‘Verna’ cultivars (each non-patented in the United States). The illustrated plants were grown under the same conditions at the Murcia region of Spain.

FIG. 1 shows the overall relatively dense and spreading growth habit of a typical specimen of the new cultivar. The photograph depicts a tree of approximately seven years of age and was obtained during March 2015.

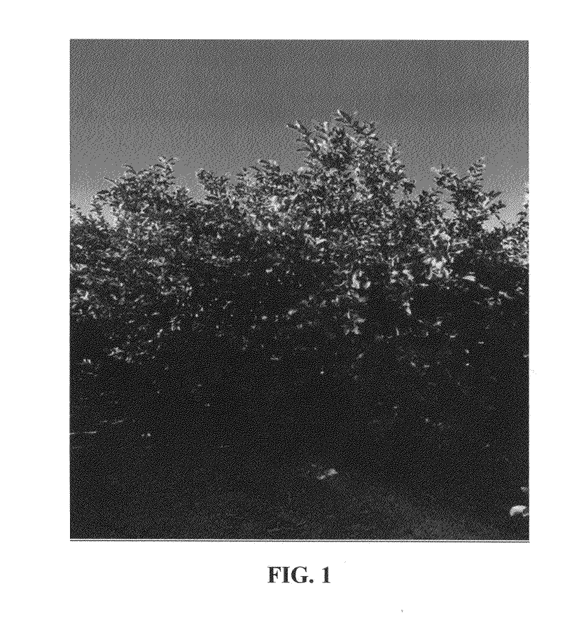

FIG. 2 shows a closer view of the foliage and the abundant formation of immature fruit that is in process of ripening while present on a typical specimen of the new cultivar. The same or a comparable tree to that of FIG. 1 is being depicted.

FIG. 3 shows whole and in cross sections attractive typical specimens of the mature fruit of the new cultivar of the invention at the time of initial harvest (i.e., typically during the middle of May).

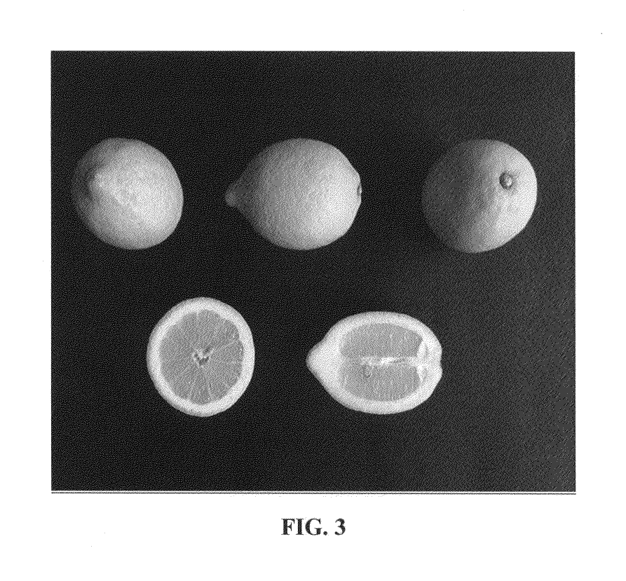

FIG. 4 shows for comparative purposes typical whole and sectioned fruit of the ‘Verna’ cultivar (top), the ‘Primofiori’ cultivar (middle), and the new cultivar of the present invention (bottom). The photograph was obtained on Feb. 13, 2015 at a time when the fruit of the new cultivar had not yet ripened to a stage of full maturity. The illustrated fruit of each cultivar was picked on the same date and was produced in the same field.

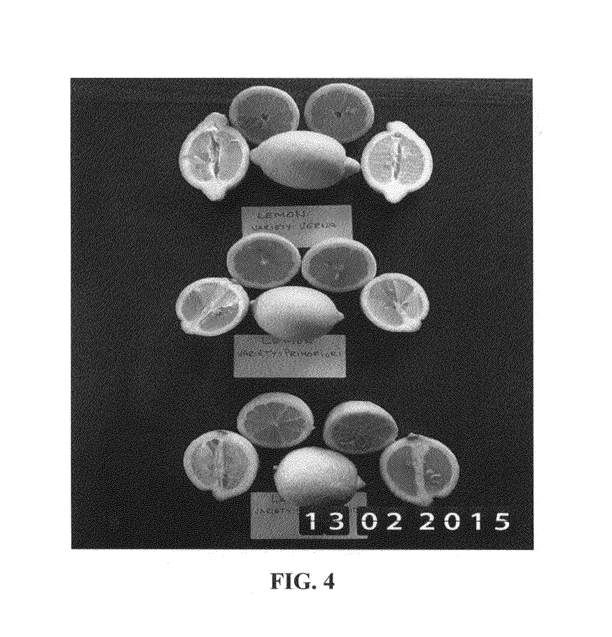

### DETAILED BOTANICAL DESCRIPTION

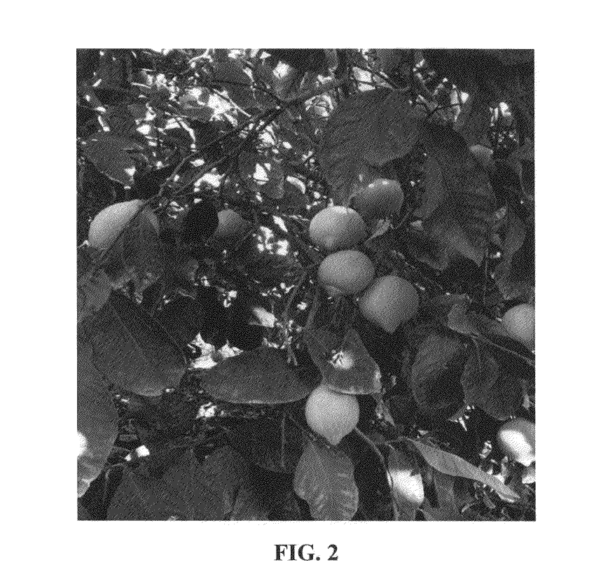

The described plants of the new cultivar were appropriately seven years of age, had been asexually reproduced by grafting on standard ‘sour orange’ rootstock (non-patented in the United States) outdoors in full sunlight at the Murcia region of Spain. Observations obtained during the growing season of 2014 are presented.
Classification: Citrus limon (L.) Burm.f.
Tree:
   Ploidy.—diploid.
   Growth habit.—dense and spreading.
   Spines.—relatively short and commonly present in a moderate quantity.
Foliage:
   Leaf shape.—generally ovate to elliptic.
   Leaf apex.—acute with the absence of emargination. leaf base substantially obtuse. leaf size relatively long and broad as illustrated in FIG. 2, and commonly intermediate in thickness.
   Leaf margin.—entire with absent or weak undulation.
   Leaf color.—commonly medium green with a moderate presence of anthocyanin.
   Petioles.—commonly medium in length with the absence of wings.
Inflorescence:
   Habit.—commonly flowers once per season.
   Buds.—commonly display a moderate presentation of anthocyanin coloration.
   Flower size.—medium in overall size.
   Petals.—commonly medium in length and width.
   Stamen.—filaments are medium in length, anthers are light-yellow in coloration and commonly bear pollen of light-yellow coloration.
   Styles.—commonly are medium in length.
Fruit:
   Configuration.—generally elongated, relatively large in diameter and broadest towards the middle region (as illustrated).
   Neck.—absent.
   Proximal end.—slightly rounded.
   Distal end.—slightly rounded with the absence of grooves.
   Nipple.—commonly present and medium in size.
   Surface texture.—generally smooth in the absence of extreme roughness, and commonly with moderate glossiness.
   Persistence of style.—commonly none.
   Rind.—commonly medium in thickness with medium oiliness.
   Surface color.—commonly light-yellow (as illustrated) in the substantial absence of variegation.
   Filling of interior.—very dense commonly with a moderate number of developed segments and the absence of rudimentary segments.
   Core diameter.—relatively small.
   Seeds.—commonly approximately 6 to 10 per fruit on average.
   Juiciness.—high.
   Maturity.—very late.
   Harvest time.—commonly during about mid-May until about mid-July in the northern Hemisphere.
   Market.—fresh for consumption, use in beverages, and for use in cooking.

Plants of the new ‘Summer Prim’ cultivar have not been observed under all possible environment conditions to date. Accordingly, it is possible that the phenotypic expression may vary somewhat with changes in light intensity and duration, cultural practices, and other environmental conditions.

I claim:

1. A new and distinct cultivar of lemon tree having the following combination of characteristics:
(a) displays diploidy,
(b) forms attractive fruit which when compared to the ‘Primofiori’ cultivar displays a substantially later season of maturity, and
(c) displays a relatively dense spreading growth habit; substantially as illustrated and described.
